(19) United States
(12) Patent Application Publication
(10) Pub. No.: US 2008/0015255 A1
(43) Pub. Date: Jan. 17, 2008

(54) Pharmaceutical Compositions Based on Fluorinated Sulphamides and Sulphinimides

(75) Inventors: Hubert Blancou, Puissalicon (FR); Franck Guillen, Montady (FR); Zohra Benfoddda, Montpellier (FR)

Correspondence Address: YOUNG & THOMPSON, 745 SOUTH 23RD STREET, 2ND FLOOR, ARLINGTON, VA, 22202, US

(73) Assignee: CENTRE NATIONAL DE LA RECHERCHE SCIENTIFIQUE(C.N.R.S.), Paris (FR)

(21) Appl. No.: 11/793,412

(22) PCT Filed: Dec. 20, 2005
(86) PCT No.: PCT/FR05/03202

§ 371 Date: Jul. 3, 2007

(30) Foreign Application Priority Data
Dec. 21, 2004 (FR) 0413676

(51) Int. Cl. A61K 31/16; A61K 31/18; A61P 27/06; A61P 31/02; A61P 43/00; A61P 9/00

(52) U.S. Cl. 514/600; 514/601

(57) ABSTRACT

Pharmacuetical composition comprising compounds of formula (I):
NZ₁Z₂Z₃ (I) in which:

Z₁, Z₂, Z₃ each independently of the others represents: a hydrogen atom;

C₁-C₆-alkyl group;

a group —SO₂R₃ wherein R₃ represents a linear or branched C₁-C₁₂-alkyl, -alkenyl or -alkynyl group, a C₃-C₁₀-cycloalkyl group or a C₆-C₁₀-aryl group, a (C₁-C₆)-alkyl-(C₆-C₁₄)-aryl group, or a C₅-C₁₀-heteroaryl group;

it being understood that at least one of the groups Z₁, Z₂, Z₃ represents a group of formula (II)
X—R_{F}—(CH₂)ₙ—SO₂— (II)

X, RF and n being as defined in claim 1.

The present invention relates to pharmaceutical compositions comprising fluorinated sulfamide and sulfonimide derivatives and to the use of fluorinated sulfamides and sulfonimides as metal enzyme inhibitors.

Many fluorinated compounds are known as medicaments, for example fluorocorticoids, such as flurbiprofen and niflumic acid. Also known are fluorinated neuroleptics of the phenothiazine type, fluorinated antidepressants such as fluoxetine, and fluorinated anxiolytics such as fluorinated benzodiazepines.

Some fluorinated amphiphilic molecules are also becoming remarkably widespread in the field of antibiotics, such as, for example, floxacillin and ofloxacin, norfloxacin and ciprofloxacin.

Glaucoma is an ocular disease that is very widespread among the world population; in France it affects 2% of people aged over 40. It is the second cause of blindness in the world after cataracts.

Glaucoma is a disease of the optic nerve caused by an increase in intraocular pressure. It manifests itself as a loss of visual field due to destruction of the optic fibres and can lead to blindness if effective treatment is not indicated. There is at present no means of curing glaucoma, but treatments intended to limit the progression of the disease.

Several classes of medicaments can be used to treat glaucoma. There may be mentioned by way of example the sympathomimetics, alpha-2-adrenergics, beta blockers, direct parasympathomimetics, or acetylcholinesterase inhibitors, docasanoids, or prostaglandins F2 alpha. Also known are carbonic anhydrase inhibitors such as acetazolamide.

However, all these medicaments have undesirable side-effects, such as allergies, coloured vision, headaches, burns or even reductions in cardiac rhythm. The side-effects are particularly serious in the case of administration by the general route.

It has been reported that trifluoromethanesulfonamide may be valuable as a carbonic anhydrase inhibitor (Maren et al., J. Biol. Chem., Vol. 268, N° 35, pages 26233-26239, 1993) owing to its solubility in water coupled with a low acid dissociation constant (Kₐ).

Document EP 0 277 814 describes the use of trifluoromethanesulfonamide and its pharmaceutically acceptable salts in the topical treatment of glaucoma. However, the compound has the disadvantage of being highly toxic. In addition, it is extremely acidic and hence difficult to formulate for topical application to the eye.

It has been found, unexpectedly, that some fluorinated sulfamides and their derivatives have an inhibiting effect on metallo-enzymes that is comparable with, or even superior to, that of acetazolamide, while having a tolerable level of toxicity.

The object of the present invention is, therefore, to propose a pharmaceutical composition comprising novel high-performance metallo-enzyme-inhibiting agents that have a tolerable level of toxicity, are soluble in water, and have low acidity or are even neutral.

A particular object of the invention is to propose the use of such compounds in the preparation of a medicament for use in pathologies in which metallo-enzymes are involved.

Those and other objects can be achieved by the present invention, which relates principally to pharmaceutical compositions comprising, in a pharmaceutically acceptable carrier, compounds of the general formula (I) below:
NZ₁Z₂Z₃ (I)
in which:

Z₁, Z₂, Z₃ each independently of the others represents:

a hydrogen atom;

a C₁-C₆-alkyl group;

a group —SO₂R₃ wherein R₃ represents a linear or branched C₁-C₁₂-alkyl, -alkenyl or -alkynyl group, a C₃-C₁₀-cycloalkyl group or a C₆-C₁₀-aryl group, a (C₁-C₆)-alkyl-(C₆-C₁₄)-aryl group, or a C₅-C₁₀-heteroaryl group;

it being understood that at least one of the groups Z₁, Z₂, Z₃ represents a group of formula (II)
X—R_{F}—(CH₂)ₙ—SO₂— (II)

in which

X represents a hydrogen atom; a fluorine atom; a group —SO₂NR₁R₂ wherein R₁, R₂ may be identical or different and each independently of the other represents a hydrogen atom, a C₁-C₆-alkyl group, a pharmaceutically acceptable cation selected from alkali metal or alkaline earth metal cations, ammonium or protonated or quaternized amines; or a group —SO₂—R₃;

R_{F} represents a linear or branched, poly- or per-fluorinated C₁-C₁₂-alkylene group;

n represents an integer from 0 to 6, it being understood that when n=0, one of the groups Z₁, Z₂, Z₃ may represent a pharmaceutically acceptable cation selected from the alkali metal or alkaline earth metal cations, ammonium or protonated or quaternized amines,

and the pharmaceutically acceptable salts of those compounds,

with the exception of compounds of formula (I) in which n=0 and at least one of the groups Z₁, Z₂, Z₃ represents CF₃.

Sulfonamide or Sulfamide Derivatives

Z₂ preferably represents a hydrogen atom.

Z₃ preferably represents a hydrogen atom.

According to a variant, when n>0, i.e. when n represents an integer from 1 to 6, the compounds of formula (I) can include the corresponding pharmaceutically acceptable organic or mineral acid salts.

Examples of acid addition salts are especially the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptanate, lactobionate, sulfamates, malonates, salicylates, propionates, methylenebis-b-hydroxynaphthoates, gentisic acid, isethionates, di-p-toluoyltartrates, methanesulfonates, ethanesulfonates, benzenesulfonates, p-toluenesulfonates, cyclohexylsulfamates and quinates-laurylsulfonate, and the like. Reference may be made in this connection to S. M. Berge et al. “Pharmaceutical Salts” J. Pharm. Sci, 66: p. 1-19 (1977).

Special preference is given to the sulfonamide derivatives, also called sulfamides, of formula (Ia):
X—R_{F}—(CH₂)ₙ—SO₂NH₂ (Ia)

which correspond to the compounds of formula (I) in which Z₁ is a compound of formula (II), X, R_{F} and n being as defined hereinbefore, and Z₂=Z₃=H.

According to a preferred variant, X represents a fluorine atom.

R_{F} preferably represents a linear poly- or per-fluorinated alkylene group.

R_{F} preferably represents a perfluorinated alkylene group having more preferably from 6 to 12 carbon atoms and yet more preferably from 6 to 8 carbon atoms.

According to a particular embodiment, R_{F} represents a perfluorinated C₂-, C₃- or C₄-alkylene group.

n preferably represents an integer from 0 to 4 and is more preferably 0 or 2.

Double Sulfamide or Sulfamide Anion Derivatives

According to another embodiment, X represents a group SO₂NR₁R₂.

When n represents an integer from 0 to 6, R₁, R₂ and/or Z₂ and Z₃ preferably each represents a hydrogen atom, then the compound of formula (I) represents a double sulfamide, also called a disulfamide.

When n=0, R₁, R₂ and/or Z₂ and Z₃ can, according to another preferred variant, each represent a hydrogen atom and an alkali metal or alkaline earth metal cation.

When X represents a group SO₂NR₁R₂, R_{F} preferably represents a perfluorinated C₂- to C₆-alkylene radical.

Sulfonamide Anion or Sulfamide Anion Derivatives

According to another preferred variant, n=0 and Z₃ represents a pharmaceutically acceptable cation selected from the alkali or alkaline earth ions, ammonium or protonated or quaternized amine.

Special preference is given to the sulfamide anion compounds of formula (Ib):
X—R_{F}—SO₂NH⁻Z₃⁺ (Ib)

As examples of alkali metal or alkaline earth metal cations there may be mentioned especially sodium, potassium, magnesium and lithium.

Examples of ammonium cations include especially ammonium (NH₄⁺) or the protonated or quaternized forms of the following organic amines: morpholine, benzathine (PhCH₂NHCH₂CH₂ NHCH₂Ph), choline hydroxide, diethanolamine, ethylenediamine, meglumine (HOCH₂CH(OH)CH(OH)CH(OH)CH(OH)—CH₂NHCH₃), procaine, N-methylpiperazine, or tromethamine ((HOCH₂)₃CNH₂).

Sulfinimide Derivatives

According to another preferred variant, Z₁ and Z₂ are identical or different and each represents a group of formula (II).

Special preference is given to the compounds of formula (Ic) below:

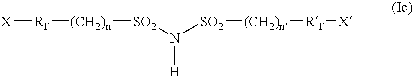

in which X, X′, R_{F}, R′_{F} and n, n′ are identical or different and are as defined hereinbefore.

Z₁ and Z₂ are preferably identical.

Mixed Sulfinimide Derivatives

According to a variant, Z₂ represents a group —SO₂R₃ wherein R₃ is as defined hereinbefore.

Preference is given to the mixed sulfinimide compounds of formula (Id) wherein Z₃=H:

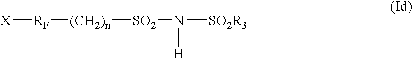

Sulfinimide Anion Derivatives

According to another variant, n=0 and Z₃ represents a pharmaceutically acceptable cation selected from the alkali metal, alkaline earth metal or ammonium cations.

According to another variant, therefore, preference is given to the sulfinimide anion derivatives represented by formulae (Ie) and (If):
X—R_{F}—SO₂—N⁺—SO₂—R′_{F}—X′, Z₃⁻ (Ie)
X—R_{F}—SO₂—N⁺—SO₂R₃, Z₃⁻ (If)

The compounds of formula (I) preferably have at least 10 fluorine atoms.

The pharmaceutical compositions according to the present invention preferably comprise the compounds of formula (I) below:
C₈F₁₇SO₂NH⁻⁺Na
C₈F₁₇SO₂NH⁻⁺Li
C₈F₁₇SO₂NH₂
C₇F₁₅SO₂NH₂
C₆F₁₃SO₂NH₂
C₈F₁₇(CH₂)₂SO₂NH₂
C₆F₁₃(CH₂)₂SO₂NH₂
(C₂F₄SO₂NH₂)₂
(C₆F₁₃SO₂)₂NH
(C₈F₁₇SO₂)₂NH
(C₄F₉SO₂)₂NH
(C₈F₁₇SO₂)NH(C₆F₁₃SO₂)
(C₆F₁₃SO₂)NH(C₄F₉SO₂)
(C₄F₉SO₂)NH(C₈F₁₇SO₂).

In this description, the term “alkyl” denotes linear- or branched-chain saturated hydrocarbon radicals having from 1 to 12 carbon atoms, preferably from 1 to 6 carbon atoms.

In the case of linear radicals, special mention may be made of the radicals methyl, ethyl, propyl, butyl, pentyl, hexyl, octyl, nonyl, decyl, dodecyl, hexadecyl and octadecyl.

In the case of branched radicals or radicals substituted by one or more alkyl radicals, special mention may be made of the radicals isopropyl, tert.-butyl, 2-ethylhexyl, 2-methylbutyl, 2-methylpentyl, 1-methylpentyl and 3-methylheptyl.

“Alkylene” denotes a branched or linear bivalent hydrocarbon chain having from 1 to approximately 6 carbon atoms. Preferred alkylene groups are lower alkylene groups having from 1 to 6 carbon atoms. Typical examples of alkylene groups include methylene and ethylene.

“Poly- or per-fluorinated alkylene” denotes an alkyl radical as defined hereinbefore substituted by at least one fluorine atom. “Perfluorinated alkylene” groups denote a branched or linear bivalent carbon chain having the formula CₙF₂ₙ— wherein n represents an integer ranging from 1 to 12.

Alkenyl radicals denote linear- or branched-chain hydrocarbon radicals and include one or more unsaturated ethylene bonds. Among the alkenyl radicals, special mention may be made of the allyl or vinyl radicals.

Alkynyl radicals denote linear- or branched-chain hydrocarbon radicals and include one or more unsaturated acetylene bonds. Among the alkynyl radicals, special mention may be made of acetylenyl.

The cycloalkyl radical is a non-aromatic, saturated or partially unsaturated mono-, bi- or tri-cyclic hydrocarbon radical having from 3 to 10 carbon atoms, such as, especially, cyclopropyl, cyclopentyl, cyclohexyl or adamantyl, as well as the corresponding rings containing one or more unsaturated bonds.

Aryl denotes a mono- or bi-cyclic aromatic hydrocarbon system having from 6 to 10 carbon atoms.

Among the aryl radicals, special mention may be made of the phenyl or naphthyl radical, more especially substituted by at least one halogen atom.

Among the alkylaryl radicals, special mention may be made of the benzyl or phenethyl radical.

Heteroaryl radicals denote mono- or bi-cyclic aromatic systems having from 5 to 10 carbon atoms and containing one or more hetero atoms selected from nitrogen, oxygen and sulfur. Among the heteroaryl radicals, mention may be made of pyrazinyl, thienyl, oxazolyl, furazanyl, pyrrolyl, 1,2,4-thiadiazolyl, naphthyridinyl, pyridazinyl, quinoxalinyl, phthalazinyl, imidazo[1,2-a]pyridine, imidazo[2,1-b]thiazolyl, cinnolinyl, triazinyl, benzofurazanyl, azaindolyl, benzimidazolyl, benzothienyl, thienopyridyl, thienopyrimidinyl, pyrrolopyridyl, imidazopyridyl, benzoazaindole, 1,2,4-triazinyl, benzothiazolyl, furanyl, imidazolyl, indolyl, triazolyl, tetrazolyl, indolizinyl, isoxazolyl, isoquinolinyl, isothiazolyl, oxadiazolyl, pyrazinyl, pyridazinyl, pyrazolyl, pyridyl, pyrimidinyl, purinyl, quinazolinyl, quinolinyl, isoquinolyl, 1,3,4-thiadiazolyl, thiazolyl, triazinyl, isothiazolyl, carbazolyl, as well as the corresponding groups obtained by fusion thereof or by fusion with the phenyl ring. Preferred heteroaryl groups include thienyl, pyrrolyl, quinoxalinyl, furanyl, imidazolyl, indolyl, isoxazolyl, isothiazolyl, pyrazinyl, pyridazinyl, pyrazolyl, pyridyl, pyrimidinyl, quinazolinyl, quinolinyl, thiazolyl, carbazolyl, thiadiazolyl, and the groups obtained by fusion with a phenyl ring, more especially quinolinyl, carbazolyl, thiadiazolyl.

“Pharmaceutically acceptable cation” and “pharmaceutically acceptable carrier” are understood as meaning a cation or carrier that is suitable for use in contact with human cells and with lower animal cells without inducing toxicity, irritation, allergic response or the like, and are commensurate with a tolerable advantage/risk ratio.

Process for the Preparation of Compounds of Formula (I)

The compounds of the general formula (I) can be prepared by applying or adapting any methods that are known per se and/or that are within the scope of the person skilled in the art, especially the method described by Larock in Comprehensive Organic Transformations, BCH Pub., 1989, or by applying or adapting the processes described in the following examples.

More particularly, the sulfamide compounds can be prepared by a process comprising the following steps:

a1) converting a compound of formula (III) into a compound of formula (IV):

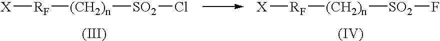

b1) bringing the compound of formula (IV) into contact with HNZ₂Z₃ to yield a compound of formula (I):

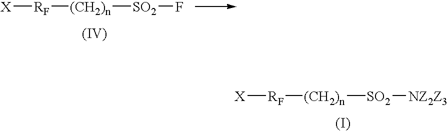

wherein X, R_{F}, n, Z₂ and Z₃ are as defined hereinbefore.

Step a1)

The step of replacing the chlorine atom by a fluorine atom can be carried out according to known methods.

Examples of suitable fluorination processes are especially:

the process of electrochemical fluorination (ECF) described in U.S. Pat. No. 2,732,398;

the process of nucleophilic substitution of the chlorine in a dissociating solvent (amide or sulfolane form) (S. Bénéfice-Malouet, H. Blancou, R. Teissèdre and A. Commeyras, Journal of Fluorine Chemistry, 31 (1986) 319-332), or alternatively

the process of substitution of the chlorine in a biphasic medium in the presence of a fluorinating agent formed of an aminated compound and ammonium bifluoride (WO 02/081081).

When n≠0, the step of replacing the chlorine by fluorine according to step a1) is preferably carried out in the presence of an alkali metal fluoride such as potassium fluoride, in an acidic medium, especially in glacial acetic acid.

Step b1)

The replacement of the fluorine atom by NZ₂Z₃ can be carried out by conventional methods.

Preparation of the Compounds of Formula (III)

According to a preferred variant, the compounds of formula (III) in which n=0 are prepared according to the process described in patent application WO 02/081431, which comprises the following steps:

a2) bringing a compound X—R_{F}-Hal, wherein Hal represents a halogen atom and X, R_{F} are as defined hereinbefore, into contact with an alkali metal hydrogen sulfite in the presence of at least one alkali metal or alkaline earth metal hydroxide;

b2) chlorinating the resulting intermediate to yield a compound of formula (III).

For the preparation of disulfamides, the procedure is carried out under the same conditions using a compound Hal-R_{F}-Hal instead of the compound X—R_{F}-Hal.

Step a2)

The compounds X—R_{F}-Hal and Hal-R_{F}-Hal are known products or can be prepared according to known methods. By way of example, n-perfluoroalkyl iodides are available commercially.

An example of an alkali metal hydrogen sulfite is sodium hydrogen sulfite (Na₂S₂O₄).

As examples of alkali metal or alkaline earth metal hydroxides that can be used according to the invention there may be mentioned especially LiOH, NaOH, KOH, Ba(OH)₂ and Ca(OH)₂.

Step b2)

The chlorination step according to step b2) can be carried out by any known method, for example with gaseous Cl₂, especially in water.

According to another variant, the poly- or per-fluoroalkanesulfonyl fluorides of formula (IV) are prepared starting from the corresponding alkanesulfonyl fluorides according to a process comprising an electrochemical fluorination (ECF) step:

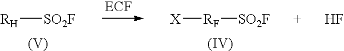

wherein X═F, R_{F} being as defined hereinbefore and R_{H} denoting a linear or branched C₁-C₁₂-alkyl group.

The fluorination process has been described especially in U.S. Pat. No. 2,732,398.

The alkanesulfonyl fluoride compounds can be prepared according to the process comprising a step of converting the alkanesulfonyl chloride of formula (IV) into alkanesulfonyl fluoride of formula (V):

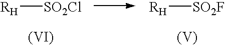

in which R_{H} represents a C₁-C₁₂-alkyl group.

According to a variant, the compounds of formula (III) can be prepared according to the process comprising:

a3) converting the compound X—R_{F}—(CH₂)ₙ—I into X—R_{F}—(CH₂)ₙ—SCN;

b3) converting the compound X—R_{F}—(CH₂)ₙ—SCN into X—R_{F}—(CH₂)ₙ—Cl.

That synthesis route is particularly suitable for the compounds of formula (III) in which n≠0.

The compounds X—R_{F}—(CH₂)ₙ—I are available commercially or can be prepared according to known methods.

Steps a3) and b3) are reactions known to the person skilled in the art and can be carried out according to conventional methods. Reference may be made in this connection to the work of March, Jerry, Advanced Organic Chemistry, 3^{rd} Ed., John Wiley and Sons.

By way of example, step a3) is carried out in the presence of an alkali metal thiocyanate, such as potassium thiocyanate, in an acidic organic medium.

By way of illustration, step b3) can be carried out in the presence of a chlorinating agent, such as sulfuryl chloride.

Process for the Preparation of Sulfamide Anions

The compounds of formula X—R_{F}—SO₂—NH⁻Z₃⁺(Ib) can be prepared according to the process comprising:

a4) bringing a compound X—R_{F}—SO₂—NH₂ into contact with a base BZ₃ in a solvent, and optionally

b4) recovering the resulting compound of formula (Ib).

The sulfamide compound of formula X—R_{F}—SO₂—NH₂ (Ia) can be prepared according to steps a1) and b1) mentioned above. There is no particular restriction regarding the nature of the base to be used in that reaction, and any base conventionally employed in reactions of this type can also be used here, on condition that it does not have an undesirable effect on the other parts of the molecule. Examples of suitable bases include alkali metal hydrides, such as sodium hydrides and potassium hydrides, alkyllithium compounds, such as methyllithium and butyllithium, alkali metal alcoholates, such as sodium methoxide and sodium ethoxide, alkali metal carbonates, such as sodium carbonate.

The base BZ₃ is preferably selected from CH₃OZ₃, CO₃(Z₃)₂, Z₃OH, NH₂Z₃.

Process for the Preparation of Sulfinimides and Sulfinimide Anions

The sulfinimides can be prepared according to known methods. In particular, the sulfinimides of formula X—R_{F}—SO₂—NH—SO₂—R_{F}′—X′ and the corresponding sulfinimide anions can be prepared especially according to the method described in the publication DesMarteaux et al. (Li-quing Hu; Darryl D. Desmarteau; Inorg. Chem, 1993, 32, 5007-5010).

That method comprises:

a5) reacting a sulfamide anion compound X—R_{F}—SO₂—NH⁻Z₃⁺(Ib) and a hexaalkyldisilazane, yielding a siliceous intermediate (X—R_{F}—SO₂)(Si(alk)₃)N⁻Z₃⁺ wherein alk denotes an alkyl group;

b5) reacting the siliceous intermediate with a compound X′—R_{F}′—SO₂—F, yielding a sulfinimide anion (X—R_{F}—SO₂)(X′—R_{F}′—SO₂)N⁻Z₃⁺, and optionally

c5) subjecting the resulting sulfinimide anion compound to acid hydrolysis, yielding a sulfinimide (X—R_{F}—SO₂)—NH—(SO₂—R_{F}′—X′).

X and X′ preferably represent a fluorine atom.

The acids which can be used for the acid hydrolysis are especially mineral acids, such as hydrochloric acid, sulfuric acid, nitric acid and phosphoric acid; and sulfonic acids, such as methanesulfonic acid, ethanesulfonic acid, benzenesulfonic acid and p-toluenesulfonic acid.

The preparation of the sulfinimides in which n≠0 can be carried out according to conventional methods. Reference may be made in this connection to the work of March, Jerry, Advanced Organic Chemistry, 3^{rd} Ed., John Wiley and Sons.

In the reactions described above, it may be necessary to protect the reactive functional groups when they are desired in the final product, or to avoid their undesirable participation in the reactions. Conventional protecting groups can be used in accordance with standard practice, for examples see T. W. Green and P. G. M. Woets in Protective Groups in Organic Chemistry, John Wiley and Sons 1991; J. F. W. Macomy in Protective Groups in Organic Chemistry, Plenum Press 1973).

The compounds so prepared can be recovered from the reaction mixture by conventional means. For example, the compounds can be recovered by removing the solvent from the reaction mixture by distillation or, if necessary, after distillation of the solvent from the mixture of the solution, by pouring the remainder into water, followed by extraction with a water-immiscible organic solvent, and by removing the solvent from the extract by distillation. Furthermore, the product can, if desired, be purified further by various techniques, such as recrystallisation, precipitation or the various chromatographic techniques, especially column chromatography or preparative thin-layer chromatography.

It will be understood that the compounds used according to the present invention can contain an asymmetric centre. Such asymmetric centres can be in the R or S configuration, independently. It will be apparent to the person skilled in the art that some compounds used according to the invention can likewise have geometric isomerism. It must be understood that the present invention includes individual geometric isomers and stereoisomers and mixtures thereof, including racemic mixtures of compounds of formula (I) above. Those types of isomers can be separated from their mixture by applying or adapting known processes, for example chromatography or stabilisation techniques, or they are prepared in isolation starting from the appropriate isomers of their intermediate.

The base products or reagents used are available commercially and/or can be prepared by applying or adapting known processes, for example processes as described in the reference examples or their obvious chemical equivalents.

According to another aspect, the invention relates to the use of compounds of formula I:
NZ₁Z₂Z₃ (I)

in which:

Z₁, Z₂, Z₃ each independently of the others represents:

a hydrogen atom;

a C₁-C₆-alkyl group;

a group —SO₂R₃ wherein R₃ represents a linear or branched C₁-C₁₂-alkyl, -alkenyl or -alkynyl group, a C₃-C₁₀-cycloalkyl group or a C₆-C₁₀-aryl group, a (C₁-C₆)-alkyl-(C₆-C₁₄)-aryl group, or a C₅-C₁₀-heteroaryl group;

it being understood that at least one of the groups Z₁, Z₂, Z₃ represents a group of formula (II)
X—R_{F}—(CH₂)ₙ—SO₂— (II)

in which

X represents a hydrogen atom; a fluorine atom; a group —SO₂NR₁R₂ wherein R₁, R₂ may be identical or different and each independently of the other represents a hydrogen atom, a C₁-C₆-alkyl group, a pharmaceutically acceptable cation selected from the alkali metal or alkaline earth metal cations, ammonium or protonated or quaternized amines; or a group —SO₂—R₃;

R_{F} represents a linear or branched, poly- or per-fluorinated C₁-C₁₂-alkylene group;

n represents an integer from 0 to 6, it being understood that when n=0, one of the groups Z₁, Z₂, Z₃ may further represent a pharmaceutically acceptable cation selected from the alkali metal or alkaline earth metal cations, ammonium or protonated or quaternized amines,

and the pharmaceutically acceptable salts of those compounds,

with the exception of compounds of formula (I) in which n=0 and at least one of the groups Z₁, Z₂, Z₃ represents CF₃,

in the manufacture of a medicament or antidote for the treatment of a pathology involving the activity of a metallo-enzyme, in particular for the treatment of a pathology for which metallo-enzyme-inhibiting activity is desirable.

The metallo-enzymes are more particularly selected from carbonic anhydrase, botulic toxin, tetanic toxin, bacterial elastase, integrase and angiotensin converting enzyme and the lethal factor of carbon.

Carbonic anhydrase is a zinc (Zn²⁺) enzyme which catalyses the hydration of carbon dioxide and the dehydration of bicarbonate. It is involved in cell respiration. The inhibition of that enzyme by the compounds according to the invention is particularly useful in the treatment of glaucoma, especially by the topical route.

Botulic toxin, anthrax toxin and tetanic toxin are also zinc (Zn²⁺) enzymes which have the effect of inducing paralysis at a very small dose. The inhibition of those toxins by the compounds according to the invention is particularly advantageous as an antidote and might be useful as a means of protection in the case of bacteriological warfare.

Bacterial elastase is a zinc (Zn²⁺) enzyme which has the effect of destroying tissues and in particular in the case of Pseudomonas aeruginosae of facilitating necroses. The inhibition of that enzyme by the compounds according to the invention is particularly useful for the treatment, especially the topical treatment, of wounds and scars, as well as for the treatment of superinfections, in particular in mucoviscidosis.

Integrase is a Mg²⁺ enzyme which permits the penetration of the DNA of HIV into the cell nucleus. The inhibition of that enzyme by the compounds according to the inverition can be used in the treatment of AIDS.

Angiotensin converting enzyme is a zinc (Zn²⁺) enzyme which plays an important role in the kidneys and in vascular regulation. The inhibition of that enzyme by the compounds according to the invention can be used in the pathogenesis of cardiovascular diseases, in particular in the treatment of arterial hypertension.

The compounds of formula (I) are therefore useful in the treatment of pathologies associated with the activity of those metallo-enzymes, such as glaucoma, mucoviscidosis, AIDS and cardiovascular diseases, especially arterial hypertension.

The compounds of formula (I) can additionally be used in the preparation of medicaments for use as antidotes to metallo-enzymes that are toxins, such as especially botulic toxin, anthrax toxin and tetanic toxin.

The invention relates preferably to the use of the compounds of formula (I) in the manufacture of a medicament for the treatment of glaucoma.

The pharmaceutical compositions according to the invention can be in forms that are intended especially for administration by the parenteral, oral, topical or ocular route.

The topical or ocular route is particularly preferred for the treatment of glaucoma.

For topical or ocular administration, the pharmaceutical composition can be in the form of a cream, an ointment, a gel or a collyrium.

The pharmaceutical composition according to the invention can be in the form of injectable solutions or suspensions in multidose vials, in liquid, pasty or solid form, and more particularly in the form of creams, milks, ointments, powders, imbibed buffers, solutions, gels, spray, foam, suspension or solution.

The dosage can vary within constant limits depending on the therapeutic indication and the route of administration, as well as on the age and weight of the patient.

The examples which follow illustrate the invention without limiting it. The starting materials used are known products prepared according to known procedures.

### EXAMPLES

The ¹H NMR and ¹⁹F NMR spectra were recorded at ambient temperature on BRUCKER AC 250 MHz and 300 MHz devices, respectively.

Chemical shifts are expressed in parts per million (ppm), the multiplicity of the signals is indicated by one (or more) lower case letter(s): s (singlet), d (doublet), q (quadruplet), m (multiplet), l (broad).

Monitoring of enzyme kinetics was carried out by means of a KONTRON UVIKON 860 ultraviolet spectrophotometer equipped with a cell adjusted to a temperature of 25° C. Measurements were carried out at a wavelength of 348 nm, allowing the hydrolysis of the substrate (para-nitrophenyl acetate) in corresponding para-nitrophenol and in acetic acid to be monitored.

pH measurements were carried out by means of a Hanna pH 213 pH meter.

### Synthesis of the sulfamides in perfluorinated series (Examples 1 and 2)

The fluorinated precursors of the various sulfamides (perfluoroalkanesulfonyl fluoride R_{F}SO₂F) were synthesized according to the method described in patents FR04819 and FR04821.

Into a 500 ml autoclave containing 1 equivalent of perfluoroalkanesulfonyl fluoride there is introduced, at ambient temperature and under pressure (80 psi), an amount greater than 3 equivalents of ammonia.

When the reaction is complete (¹⁹F NMR monitoring/solvent CDCl₃), excess ammonia which is displaced by a stream of nitrogen is neutralized by a 1N aqueous hydrochloric acid solution.

The reaction mixture is taken up in a mixture (v/v) of diethyl ether and 0.1N hydrochloric acid.

The ethereal phase containing the sulfamide is dried with sodium sulfate and then the solvent is evaporated off under reduced pressure; the expected sulfamide is thus obtained.

The necessary amounts of reagents for each sulfamide prepared are shown below.

### Example 1

### Synthesis of 1,1,2,2,3,3,4,4,5,5,6,6,7,7,8,8,8-heptadecafluoro-octanesulfamide

202.16 g (0.4 mol) of C₈F₁₇SO₂F are reacted with 1.2 mol of ammonia.

189 g of C₈F₁₇SO₂NH₂ crystals are obtained, which represents a yield of 95%.

¹⁹F NMR (CD₃OD/CFCl₃) δ (ppm): −125.6 (m, 2F, (—(CF₂)₆—CF₂—CF₃); −122 (m, 2F, —(CF₂)₅—CF₂—CF₂—CF₃); −121 (m, 6F, —(CF₂)₂—(CF₂)₃—(CF₂)₂—CF₃); −119.7 (m, 2F, —CF₂—CF₂—(CF₂)₅—CF₃); −113.4 (m, 2F, SO₂—CF₂—(CF₂)₆—CF₃); −80.7 (t, 3F, —(CF₂)₇—CF₃).

¹H NMR: (DMSO) δ (ppm): 9.040 (s, 2H, C₈F₁₇SO₂NH₂)

MS (FAB⁻, NBA): [M−H⁺]=498

Spectral comparison identical with a previously prepared authentic sample.

### Example 2

### Synthesis of 1,1,2,2,3,3,4,4,5,5,6,6,6-tridecafluoro-n-hexane-sulfamide

162 g (0.4 mol) of C₆F₁₃SO₂F are reacted with 1.2 mol of ammonia.

151 g of C₆F₁₃SO₂NH₂ crystals are obtained, which represents a yield of 95%.

¹⁹F NMR (CD₃OD/CFCl₃) δ (ppm): −125 (m, 2F, (—(CF₂)₄—CF₂—CF₃); −122 (m, 2F, —(CF₂)₃—CF₂—CF₂—CF₃); −121 (m, 4F, —CF₂—(CF₂)₂—(CF₂)₂—CF₃); −113.4 (m, 2F, SO₂—CF₂—(CF₂)₄—CF₃); −80.7 (t, 3F, —(CF₂)₅—CF₃).

¹H NMR: (DMSO) δ (ppm): 9.040 (s, 2H, C₈F₁₇SO₂NH₂)

MS (FAB⁻, NBA): [M−H⁺]=398

Spectral comparison identical with a previously prepared authentic sample.

### Disulfamide Synthesis in Perfluorinated Series

This synthesis is based on the perfluoroalkanesulfamide synthesis described in French Patents Nos. FR 01 04819 and FR 01 04821.

### Example 3

### Synthesis of perfluorobutane-1,4-disulfamide ((CF₂)₂(CF₂SO₂NH₂)₂)

### a) Synthesis of sodium perfluorobutane-1,4-disulfinate ((CF)₂(CF₂SO₂Na)₂)

22.3 g (0.050 mol) of I—(CF₂)₄—I are added, at 45° C., to a solution composed of 75 ml of water, 40 ml of acetonitrile, 6 g (0.107 mol) of quicklime (CaO) and 17.84 g (0.107 mol) of sodium dithionite (Na₂S₂O₄).

The reaction is monitored by ¹⁹F NMR of the crude reaction mixture; it is virtually instantaneous.

The reaction mixture is filtered in order to remove the residual salts, and the acetonitrile is distilled off at atmospheric pressure.

The aqueous phase which remains and which contains the perfluorinated sodium disulfinate is used directly in the following step.

### b) Synthesis of perfluorobutane-1,4-disulfonyl chloride ((CF₂)₂(CF₂SO₂Cl)₂)

The temperature of the preceding aqueous phase is brought to 0-5° C., and the sulfonyl chloride is obtained by adding 7 g (0.2 mol) of gaseous chlorine to the solution.

The temperature is then brought to 35° C. (melting point of the chlorinated product).

The product is recovered by decantation.

Crude yield: 85%, purity 90-95%.

### c) Synthesis of n-perfluorobutane-1,4-disulfonyl fluoride ((CF₂)₂(CF₂SO₂F)₂)

30 ml of an aqueous solution composed of 0.84 g (0.12 M) of ammonium bifluoride and 12.14 g (0.12 M) of triethylamine are added dropwise to 20 g (0.05 mol) of (CF₂)₂(CF₂SO₂Cl)₂ dissolved in 15 ml of dichloromethane; the temperature of the medium must not exceed 18-20° C.

After one hour's reaction, the organic phase containing the sulfonyl fluoride is recovered by decantation; that phase is dried and used directly in the following step.

Crude yield 85%.

### d) Synthesis of perfluorobutane-1,4-disulfamide ((CF₂)₂(CF₂SO₂NH₂)₂)

0.05 mol of ammonia is added slowly to 10 ml of the preceding solution containing 4 g (0.011 mol) of ((CF₂)₂(CF₂SO₂F)₂; the temperature of the reaction medium is maintained at 5° C.

When the reaction is complete (¹⁹F NMR monitoring), the solvent is evaporated off under reduced pressure and the resulting crystals are taken up in a mixture of dilute hydrochloric acid and ethyl acetate.

The organic phase is dried and the solvent is evaporated off under reduced pressure.

3 g of sulfamide are obtained, which represents a crude yield of 75%.

¹⁹F NMR (DMSO): δ (ppm): −120.09 (m, 4F, H₂NSO₂—CF₂—(CF₂)₂—CF₂—SO₂NH₂); −113.77 (m, 4F, H₂NSO₂—CF₂—(CF₂)₂—CF₂—SO₂NH₂)

### Synthesis of the Fluorohydrogenated Sulfamides

### Example 4

### Synthesis of 3,3,4,4,5,5,6,6,7,7,8,8,8-tridecafluoro-n-octane-sulfamide C₆F₁₃(CH₂)₂SO₂NH₂

### a) Synthesis of 3,3,4,4,5,5,6,6,7,7,8,8,8-tridecafluoro-n-octane thiocyanate C₆F₁₃(CH₂)₂SCN

A solution of 500 ml of ethanol containing 47.4 g (0.1 mol) of C₆F₁₃C₂H₄I and 10 g (0.16 mol) of glacial CH₃CO₂H is reacted with 14.55 g(0.15 mol) of KSCN. The reaction mixture is heated at reflux of the ethanol for 4 hours.

The ethanol is then distilled off (78° C.-0.3 mmHg) and then the temperature is lowered to 70° C. After addition of 100 ml of water, the thiocyanate is recovered by decantation.

38.475 g of product are recovered, which represents a crude yield of 95%.

¹H NMR (CDCl₃) δ (ppm): 3.4 (m, 2H, C₆F₁₃—CH₂—CH₂—SCN); 2.8 (m, 2H, C₆F₁₃—CH₂CH₂—SCN)

¹⁹F NMR (DMSO): δ (ppm): −126.47 (m, 2F, CF₃—CF₂—(CF₂)₄—(CH₂)₂SCN; −123.28 (m, 4F, CF₃—CF₂—(CF₂)₂(CF₂)₂—(CH₂)₂SCN), −122.07 (m, 2F, CF₃—(CF₂)₃—CF₂—CF₂—CH₂)₂SCN), −113.50 (m, 2F, CF₃—(CF₂)₄—CF₂—(CH₂)₂SCN, −81.32 (m, 3F, CF₃—(CF₂)₅—(CH₂)₂SCN).

### b) Synthesis of 3,3,4,4,5,5,6,6,7,7,8,8,8-tridecafluoro-n-octanesulfonyl chloride (C₆F₁₃(CH₂)₂SO₂Cl)

35.07 g (0.259 mol) of SO₂Cl₂ are added in the course of 30 minutes, at a temperature of 50° C., to 20 g (0.049 mol) of previously distilled C₆F₁₃C₂H₄SCN, and then 30 ml of acetic acid containing 2.5 g of water are added in the course of one hour.

When the addition is complete, stirring is carried out for 30 minutes and 30 ml of water are added. The organic phase is recovered by decantation.

19.99 g of product are recovered, which represents a crude yield of 90.7%.

¹H NMR (CDCl₃) δ (ppm): 2.83 (m, 2H, C₆F₁₃—CH₂—CH₂—SO₂Cl); 3.9 (m, 2H, C₆F₁₃—CH₂—CH₂—SO₂Cl).

### c) Synthesis of 3,3,4,4,5,5,6,6,7,7,8,8,8-tridecafluoro-n-octanesulfonyl fluoride C₆F₁₃(CH₂)₂SO₂F

3.9 g (0.067 mol) of KF are added to 20 g (0.447 mol) of C₆F₁₃C₂H₄SO₂Cl in 20 ml of glacial acetic acid.

The reaction mixture is heated at 50° C. for 2 hours.

The sulfonyl fluoride (C₆F₁₃C₂H₄SO₂F) is recovered by decantation.

27 g of product are recovered.

Crude yield 93.77%.

¹H NMR (CDCl₃) δ (ppm): 3.65 (m, 2H, C₆F₁₃—CH₂—CH₂SO₂F); 2.7 (m, 2H, C₆F₁₃—CH₂—CH₂SO₂F).

### d) Synthesis of 3,3,4,4,5,5,6,6,7,7,8,8,8-tridecafluoro-n-octanesulfamide C₆F₁₃(CH₂)₂SO₂NH₂

The protocol is the same as that for the sulfamides in perfluorinated series.

126 mmol of gaseous NH₃ are added to 42 mmol of C₆F₁₃(CH₂)₂SO₂F.

16.01 g of product are recovered.

Crude yield 89.3%.

¹⁹F NMR (DMSO): δ (ppm): −126.28 (m, 2F, CF₃—CF₂—(CF₂)₄—(CH₂)₂SO₂NH₂, −123.4 (m, 4F, CF₃—CF₂—(CF₂)₂(CF₂)₂—(CH₂)₂SO₂NH₂), −121.93 (m, 2F, CF₃—(CF₂)₃—CF₂—CF₂—(CH₂)₂SO₂NH₂), −113.12 (m, 2F, CF₃—(CF₂)₄—CF₂—(CH₂)₂SO₂NH₂, −81.06 (m, 3F, CF₃—(CF₂)₅—(CH₂)₂SO₂NH₂).

¹H NMR (DMSO): δ (ppm): 2.51 (m, 2H, C₆F₁₃—CH₂—CH₂—SO₂NH₂); 2.09 (m, 2H, C₆F₁₃—CH₂—CH₂SO₂NH₂).

### Synthesis of the Sulfamides in Hydrogenated Series (Examples 5 and 6)

### Example 5

### Synthesis of n-hexanesulfamide (n-C₆H₁₃SO₂NH₂)

### a) Synthesis of hexane thiocyanate (n-C₆H₁₃SCN)

20 g of 1 bromo-hexane (0.121 mol) are reacted with 14.67 g of potassium thiocyanate (0.15 mol) dissolved in 30 ml of anhydrous ethanol containing 0.6 g of glacial acetic acid.

After reaction at reflux for four hours, the ethanol is distilled off under reduced pressure (50 mm of mercury).

The products that have formed are taken up in 50 ml of water and 30 ml of ether.

The organic phase containing hexyl thiocyanate is dried with anhydrous sodium sulfate and then the solvent is evaporated off under reduced pressure.

The product is distilled (b.p. 64° C./0.11 mm Hg), allowing pure hexyl thiocyanate to be obtained (15.47 g).

Yield 89.4%.

¹H NMR (CD₃OD/CDCl₃) δ (ppm): 2.2 (m, 8H, CH₃—(CH₂)₄—CH₂—SCN); 3.2 (t, 2H, (SCN—CH₂—(CH₂)₄—CH₃); 1.15 (t, 3H CH₃—(CH₂)₄—CH₂—SCN).

### b) Synthesis of hexanesulfonyl chloride (nC₆H₁₃SO₂Cl)

34 ml of sulfuryl chloride are added in the course of 30 minutes to 10.95 g of hexyl thiocyanate, and then a mixture of 23 ml of acetic acid and 3.7 ml of water is added dropwise in the course of one hour to the remainder of the preparation.

The gas that evolves is trapped by a concentrated aqueous sodium hydroxide solution.

When the addition is complete, the reaction medium is stirred for 45 minutes.

Excess sulfuryl chloride is destroyed by addition of 15 ml of water.

The product is then extracted with 30 ml of dichloromethane.

The organic phase containing the hexanesulfonyl chloride is dried with sodium sulfate and then the solvent is evaporated off under reduced pressure.

11.34 g of hexanesulfonyl chloride are obtained.

Yield 80.2%.

¹H NMR (CD₃OD/CDCl₃) δ (ppm): 2.2 (m, 8H, CH₃—(CH₂)₄—CH₂—SO₂Cl); (t, 2H, (SO₂Cl—CH₂—(CH₂)₄—CH₃); 1.15 (t, 3H, CH₃—(CH₂)₄—CH₂—SO₂Cl).

### c) Synthesis of n-hexanesulfamide (nC₆H₁₃SO₂NH₂)

9.82 g of C₆H₁₃SO₂Cl (0.054 mol) are reacted with 0.11 mol of ammonia.

8 g of C₆H₁₃SO₂NH₂ are obtained, that is to say a crude yield of 90%.

¹H NMR (CD₃OD/CDCl₃) δ (ppm): 2.2 (m, 8H, CH₃—(CH₂)₄—CH₂—SO₂NH₂); 3.25 (t, 2H, H₂NO₂S—CH₂—(CH₂)₄—CH₃); 1.15 (t, 3H, CH₃—(CH₂)₄—CH₂—SO₂NH₂).

### Example 6

### Synthesis of n-octanesulfamide (nC₈H₁₇SO₂NH₂)

### a) Synthesis of octyl thiocyanate (nC₈H₁₇SCN)

23 g of 1-bromo-octane (0.12 mol) are reacted with 14.67 g of potassium thiocyanate (0.15 mol) dissolved in 30 ml of ethanol containing 0.6 g of acetic acid.

After reacting at reflux for 4 hours, the ethanol is distilled off under reduced pressure (50 mmHg).

The products that have formed are taken up in 50 ml of water and 30 ml of ether.

The organic phase containing the octyl thiocyanate is dried with sodium sulfate and then the solvent is evaporated off under reduced pressure.

The product is distilled (b.p. 77° C./0.11 mmHg), allowing pure octyl thiocyanate (17.3 g) to be obtained.

Yield 89%.

¹H NMR (CD₃OD/CDCl₃) δ (ppm): 2.2 (m, 12H, CH₃—(CH₂)₆—CH₂—SCN); 3.2 (t, 2H, (SCN—CH₂—(CH₂)₄—CH₃); 1.15 (t, 3H CH₃—(CH₂)₄—SCN).

### b) Synthesis of octanesulfonyl chloride (nC₈H₁₇SO₂Cl)

23 ml of sulfuryl chloride are added in the course of 30 minutes to 10 g (0.058 mol) of octyl thiocyanate, and then a mixture of 15 ml of acetic acid and 2.5 ml of water is added to the remainder of the preparation in the course of 1.5 hours. When the addition is complete, the reaction mixture is stirred for 45 minutes.

The gas that evolves is trapped by a concentrated aqueous sodium hydroxide solution in order to neutralize the various gases that evolve during the reaction.

Excess sulfuryl chloride is destroyed by addition of 15 ml of water.

The product is extracted with 30 ml of dichloromethane.

The organic phase containing the hexanesulfonyl chloride is dried with sodium sulfate and then the solvent is evaporated off under reduced pressure.

11.34 g of octanesulfonyl chloride are obtained.

Yield 90%.

¹H NMR (CD₃OD/CDCl₃) δ (ppm): 2.2 (m, 12H, CH₃—(CH₂)₆—CH₂—SO₂Cl); 3.65 (t, 2H, (SO₂Cl—CH₂—(CH₂)₆); 1.15 (t, 3H, CH₃—(CH₂)₄—SO₂Cl).

### c) Synthesis of n-octanesulfamide (nC₈H₁₇SO₂NH₂)

5 g of C₈H₁₇SO₂Cl (0.026 mol) are reacted with 0.051 mol of ammonia.

4.07 g of C₈H₁₇SO₂NH₂ are obtained, which represents a yield of 82.7%.

¹H NMR (CD₃OD/CDCl₃) δ (ppm): 2.2 (m, 12H, CH₃—(CH₂)₆—CH₂—SO₂NH₂); 3.25 (t, 2H, (H₂NO₂S—CH₂—(CH₂)₆—CH₃); 1.15 (t, 3H, CH₃—(CH₂)₆—CH₂—SO₂NH₂).

### Synthesis of Alkali Metal Sulfamide Anion (Examples 7 to 9)

### General Process for the Preparation of Sodium n-perfluoroalkylsulfamide Anion

An ethereal solution of 1.1 equivalents of perfluoroalkanesulfamide is added dropwise to 1 equivalent of sodamide dispersed in diethyl ether.

The ammonia that forms is displaced by a stream of nitrogen and then trapped by a 1N aqueous hydrochloric acid solution.

The resulting crystals are filtered off, washed with diethyl ether and then dried under reduced pressure (0.1 mm of mercury).

### Example 7

### Synthesis of sodium 1,1,2,2,3,3,4,4,5,5,6,6,7,7,8,8,8-heptadecafluoro-n-octylsulfamide anion (C₈F₁₇SO₂NH⁺Na)

15.08 g (0.0302 mol) of C₈F₁₇SO₂NH₂ are reacted with 1.071 g (0.0274 mol) of NaNH₂.

10.90 g of crystals are obtained, which corresponds to a yield of 76%.

¹⁹F NMR (CD₃COCD₃): δ (ppm): −125.9 (m, 2F, —(CF₂)₆—CF₂—CF₃); −122.4 (m, 2F, —(CF₂)₅—CF₂—CF₂—CF₃); −121.5 (m, 6F, —(CF₂)₂—(CF₂)₃—(CF₂)₂—CF₃); −120 (m, 2F, NH₂SO₂—(CF₂)—(CF₂)—(CF₂)₅—CF₃); −113.52 (m, 2F, NH₂SO₂—CF₂—(CF₂)₆—CF₃); −80.82 (t, 3F, —(CF₂)₇—CF₃).

MS (FAB⁻, NBA): [M−H⁺]=498

### Example 8

### Synthesis of sodium 1,1,2,2,3,3,4,4,5,5,6,6,6-tridecafluorosulfamide anion (C₆F₁₃SO₂NH⁻Na⁺)

8 g (0.02 mol) of C₆F₁₃SO₂NH₂ are reacted with 0.7 g (0.0181 mol) of NaNH₂.

5.44 g of crystals are obtained, which corresponds to a yield of 71%.

¹⁹F NMR (CD₃COCD₃): δ (ppm): −125.93 (m, 2F, —(CF₂)₄—CF₂—CF₃); −122.46 (m, 2F, —(CF₂)₃—CF₂—CF₂—CF₃); −121.56 (m, 2F, —(CF₂)₂—CF₂)₄—CF₃); −120.06 (m, 2F, —(CF₂)—CF₂—(CF₂)₃—CF₃); −113.55 (m, 2F, —CF₂—(CF₂)₄—CF₃); −80.83 (t, 3F, —(CF₂)₅—CF₃).

MS (FAB⁻, NBA): [M−H⁺]=398.

### Example 9

### Synthesis of the sodium salt of 1-butanesulfamide

### 1,1,2,2,3,3,4,4,4-nonafluorosulfamide (C₄F₉SO₂NH⁺Na)

2.59 g (0.00866 mol) of C₄F₉SO₂NH₂ are reacted with 0.307 g (0.00787 mol) of NaNH₂.

2.07 g of crystals are obtained, which corresponds to a yield of 81%.

¹⁹F NMR (CD₃COCD₃): δ (ppm): −125.82 (m, 2F, —(CF₂)₂—CF₂—CF₃); −121.01 (m, 2F, —(CF₂)—CF₂—CF₂—CF₃); −113.7 (m, 2F, —CF₂—(CF₂)₂—CF₃); −80.80 (t, 3F, —(CF₂)₃—CF₃).

MS (FAB⁻, NBA): [M−H⁺]=298

### Synthesis of the Sulfinimides in Perfluorinated Series (Examples 10 to 14)

The synthesis of the sulfinimides was carried out according to a method based on that of DesMarteaux et al. (Li-quing Hu; Darryl D. Desmarteau; Inorg. Chem, 1993, 32, 5007-5010).

The synthesis is carried out in three steps: synthesis of sodium n-perfluoroalkylsulfamide anion, formation of the siliceous intermediate, then reaction thereof with perfluoroalkanesulfonyl fluoride.

1. General Process for the Preparation of the Siliceous Intermediate

The sodium salt of anhydrous n-perfluoroalkylsulfamide is reacted in acetonitrile with an excess of freshly distilled hexamethyldisilazane (HMDS). The reaction mixture is heated at 110° C. for 24 hours.

The solvent and excess HMDS are removed by distillation under reduced pressure in situ.

The resulting solid is used without further purification in the final step of sulfinimide synthesis. (Spectral analyses (NMR, FAB, IR) were not carried out for lack of stability of the intermediate).

2. General Process for Obtaining the Sulfinimides

One equivalent of R_{F}SO₂N⁻(Na⁺)Si (CH₃)₃ is placed in a Teflon reactor with 1.15 equivalents of R_{F}SO₂F and a suitable amount of acetonitrile.

The reaction medium is heated at reflux of the acetonitrile for 48 hours, with stirring.

The solvent is evaporated-off under reduced pressure. The reaction mixture is taken up in a mixture of diethyl ether and 0.1N HCl. The resulting crystals are purified on a silica column (60% ethyl acetate/40% petroleum ether) and then washed with diethyl ether.

The necessary amounts of reagents for each sulfinimide prepared are shown below.

### Example 10

### Synthesis of bis(1,1,2,2,3,3,4,4,5,5,6,6,6)tridecafluoro-n-hexylsulfinimide C₆F₁₃SO₂NHSO₂C₆F₁₃

### a) Synthesis of sodium 1,1,2,2,3,3,4,4,5,5,6,6,6-tridecafluoro-n-hexyl-N-trimethylsilylsulfamide anion C₆F₁₃SO₂N⁻(Na⁺)Si(CH₃)₃

5.44 g (0.0129 mol) of C₆F₁₃SO₂NH⁻Na⁺ are reacted with 54 ml (0.258 mol) of HMDS and 17.4 ml (0.33 mol) of acetonitrile.

5.68 g of product are obtained in a yield of 89.3%.

### b) Synthesis of bis(1,1,2,2,3,3,4,4,5,5,6,6,6-tridecafluoro-n-hexylsulfinimide C₆F₁₃SO₂NHSO₂C₆F₁₃

3.42 g (0.00694 mol) of C₆F₁₃SO₂N⁻(Na⁺)Si(CH₃)₃ are reacted with 3.21 g (0.008 mol) of C₆F₁₃SO₂F and 10 ml of acetonitrile.

1.73 g of crude product are obtained, which represents a yield of 31.95%.

¹⁹F NMR (CD₃COCD₃): δ (ppm): −125.84 (m, 4F, NH(SO₂—(CF₂)₄—CF₂—CF₃)₂), −122.38 (m, 4F, —NH(SO₂—(CF₂)₃—CF₂—CF₂—CF₃)₂); −121.44 (m, 4F, NH(SO₂—(CF₂)₂—CF₂—(CF₂)₂—CF₃)₂); −119.71 (m, 4F, NH(SO₂—CF₂—CF₂(CF₂)₃—CF₃)₂); −112.83 (m, 4F, NH(SO₂—CF₂—(CF₂)₄—CF₃)₂); −80.75 (t, 6F, NH(SO₂—(CF₂)₅—CF₃)₂).

MS (FAB⁻; NBA): [M−H]⁺=780

### Example 11

### Synthesis of C₄F₉SO₂NH.SO₂C₄F₉

### a) Synthesis of sodium 1,1,2,2,3,3,4,4,4-nonadecafluoro-n-butyl-N-trimethylsilylsulfamide anion C₄F₉SO₂N⁻(Na⁺)Si(CH₃)₃

1 g (0.0031 mol) are reacted with 16.43 ml (0.0778 mol) of HMDS and 13.1 ml (0.25 mol) of acetonitrile.

1.21 g of product are obtained with a yield of 98.9%.

### b) Synthesis of C₄F₉SO₂NH.SO₂C₄F₉

1.21 g (0.0031 mol) of C₄F₉SO₂N⁻(Na+)Si(CH₃)₃ are reacted with 1.07 g (0.0035 mol) of C₄F₉SO₂F and 5.4 ml of acetonitrile.

0.8 g of crude product is obtained, that is to say a yield of 44.85%.

¹⁹F NMR (CD₃COCD₃): δ (ppm): −125.73 (m, 4F, NH(SO₂—(CF₂)₂—CF₂—CF₂—CF₃)₂); −120.71 (m, 4F, NH(SO₂—CF₂—CF₂—CF₂—CF₃)₂); −113.0 (m, 4F, NH(SO₂—CF₂—(CF₂)₂—CF₃)₂); −80.80 (t, 6F, NH(SO₂—(CF₂)₃—CF₃)₂).

MS (FAB⁻; NBA): [M−H]⁺=580

### Example 12

### Synthesis of C₆F₁₃SO₂NHSO₂C₄F₉

5.68 g (0.0115 mol) of C₆F₁₃SO₂N⁻(Na+)Si(CH₃)₃ are reacted with 4 g (0.0132 mol) of C₄F₉SO₂F and 20 ml of acetonitrile.

2.8 g of crude product are obtained, that is to say a yield of 35.75%.

¹⁹F NMR (CD₃COCD₃): δ (ppm): −125.81 (m, 4F, CF₃—CF₂—(CF₂)₄—SO₂NHSO₂—CF₂—CF₂—CF₂—CF₃); −122.46 (m, 2F, CF₃—CF₂—CF₂—(CF₂)₃—SO₂NHSO₂—CF₂—CF₂—CF₂—CF₃); −121.44 (m, 2F, CF₃—(CF₂)₂—CF₂—(CF₂)₂SO₂NHSO₂—(CF₂)₃CF₃); −120.73 (m, 2F, CF₃—(CF₂)₅—SO₂NHSO₂—CF₂—CF₂—CF₂—CF₃); −113.01 (m, 2F, CF₃—(CF₂)₄—CF₂—SO₂NHSO₂—(CF₂)₃CF₃); −112.82 (m, 2F, CF₃—(CF₂)₅—SO₂NHSO₂—CF₂—(CF₂)₂CF₃).

MS (FAB⁻; NBA): [M−H]⁺=680

### Example 13

### Synthesis of C₈F₁₇SO₂NH.SO₂C₈F₁₇

### a) Synthesis of sodium 1,1,2,2,3,3,4,4,5,5,6,6,7,7,8,8,8-heptadecafluoro-n-octyl-N-trimethylsilylsulfamide anion C₈F₁₇SO₂N⁻(Na⁺)Si(CH₃)₃

6 g (0.115 mol) of C₈F₁₇SO₂NH⁻Na⁺ are reacted with 60 ml (0.284 mol) of HMDS and 19.2 ml (0.365 mol) of acetonitrile.

6.75 g of product are obtained with a yield of 98.8%.

### b) Synthesis of C₈F₁₇SO₂NH.SO₂C₈F₁₇

6.63 g (0.0111 mol) of C₈F₁₇SO₂N⁻(Na+)Si(CH₃)₃ are reacted with 6.45 g (0.0128 mol) of C₈F₁₇SO₂F and 19.6 ml of acetonitrile.

7.5 g of crude product are obtained, that is to say a yield of 68.87%.

¹⁹F NMR (CD₃COCD₃): δ (ppm ): −125.83 (m, 4F, NH(SO₂—(CF₂)₆—CF₂—CF₃)₂); −122.36 (m, 4F, NH(SO₂—(CF₂)₅—CF₂—CF₂—CF₃)₂); −121.45 (m, 12F, NH(SO₂—(CF₂)₂—(CF₂)₃—(CF₂)₂—CF₃)₂); −112.83 (m, 2F, NH(SO₂—CF₂—(CF₂)₆—CF₃)₂); −80.75 (t, 6F, NH(SO₂—(CF₂)₆—CF₃)₂).

MS (FAB⁻; NBA): [M−H]⁺=980

### Example 14

### Synthesis of C₄F₉SO₂NHSO₂C₈F₁₇

1.18 g (0.003 mol) of C₄F₉SO₂N⁻(Na+)Si(CH₃)₃ are reacted with 1.73 g (0.0034 mol) of C₈F₁₇SO₂F and 20 ml of acetonitrile.

1.35 g of crude product are obtained, that is to say a yield of 57.62%.

¹⁹F NMR (CD₃COCD₃): δ (ppm): −125.75 (m, 4F, CF₃—CF₂—CF₂—CF₂—SO₂NH—SO₂—(CF₂)₆—CF₂—CF₃); −122.3 (m,4F, CF₃—CF₂—CF₂—CF₂—SO₂NH—SO₂—(CF₂)₅—CF₂—CF₂—CF₃); −121.37 (m, 6F, CF₃—CF₂—CF₂—CF₂—SO₂NH—SO₂—(CF₂)₃—(CF₂)₃—CF₂—CF₃); −120.72 (m, 4F, CF₃—CF₂—CF₂—CF₂—SO₂NH—SO₂—(CF₂)₇—CF₃); −119.65 (m, 4F, CF₃—CF₂—CF₂—CF₂—SO₂NH—SO₂—CF₂—CF₂—(CF₂)₅—CF₃); −112.96 (m, 2F, CF₃—CF₂—CF₂—CF₂—SO₂NH—SO₂—(CF₂)₇—CF₃); −112.87 (m, 2F, CF₃—CF₂—CF₂—CF₂—SO₂NH—SO₂—CF₂—CF₂—(CF₂)₅—CF₃); −80.78 (t, 3F, CF₃—(CF₂)₃—SO₂NH—SO₂—(CF₂)₇—CF₃); −80.74 (t, 3F, CF₃—(CF₂)₃—SO₂NH—SO₂—(CF₂)₇—CF₃).

MS (FAB⁻; NBA): [M−H]⁺=780

### Example 15

### Synthesis of C₈F₁₇SO₂NHSO₂C₆F₁₃

4.51 g (0.0076 mol) of C₈F₁₇SO₂NHSi(CH₃)₃ are reacted with 3.51 g (0.0087 mol) of C₆F₁₃SO₂F and 13.34 ml of acetonitrile.

4 g of crude product are obtained, that is to say a yield of 59.7%.

¹⁹F NMR (CD₃COCD₃): δ (ppm): −125.83 (m, 4F, CF₃—CF₂—CF₂—(CF₂)₃—CF₂—CF₂—SO₂—NH—SO₂—(CF₂—)₄—CF₂—CF₃; −122.3 (m, 4F, (CF₃—CF₂—CF₂—(CF₂)₃—CF₂—CF₂—SO₂—NH—SO₂—(CF₂)₃—CF₂—(CF₂—)—CF₃); −121.4 (m, 8F, (CF₃—CF₂—CF₂—(CF₂)₃—CF₂—CF₂—SO₂—NH—SO₂—(CF₂)₂—CF₂—(CF₂)₂—CF₃); −119.7 (m, 4F, (CF₃—(CF₂)₅—CF₂—CF₂—SO₂—NH—SO₂—CF₂—CF₂—(CF₂—)₃—CF₃); −112.82 (m, 4F, (CF₃—(CF₂)₅—CF₂—CF₂—SO₂—NH—SO₂—CF₂—CF₂—(CF₂—)₃—CF₃); −80.7 (m, 4F, CF₃—(CF₂)₇—SO₂NH—SO₂—(CF₂)₅—CF₃.

MS (FAB⁻; NBA): [M−H]⁺=880

### Example 16

### Inhibition of Bovine Carbonic Anhydrase by Sulfamides, and Kinetic Study

Carbonic anhydrase is an enzyme which obeys the Michaelis-Menten equation. That equation predicts the relationship between the rate of reaction and the concentration of the substrate, if the concentration of the enzyme is kept constant: which, in the present case, is true.

$$v = \frac{V m [ S ]}{K m + [ S ]}$$

where Vm or Vmax is the maximum rate of reaction;

where Km (Michaelis constant) is equal to the substrate concentration for which the rate is half the maximum rate;

where [S] is the substrate concentration.

a) Study Model of Carbonic Anhydrase Activity in Vitro

For this study, an adaptation of the method of Armstrong J.; Myers, D.; Verpoorte, J.; Edsall, J.; J. Biol. Chem., 1966, 241-21, 5137-5149 was used.

Carbonic anhydrase catalyzes not only the reversible hydration of CO₂ and the dehydration of HCO₃, but also the hydrolysis of numerous esters and, in particular, paranitrophenyl acetate (NPA). Carbonic anhydrase activity can therefore be studied either by monitoring on the basis of CO₂ hydration or by studying the hydrolysis of that ester.

Although that esterase activity is very weak compared with carbonic anhydrase activity, it allowed a spectrophotometric determination to be carried out, and the latter method was therefore chosen for practical reasons.

1. Principle of the Determination

The hydrolysis of para-nitrophenyl acetate by carbonic anhydrase yields 4-nitrophenol, which absorbs at a wavelength of 348 nm. Para-nitrophenyl acetate, on the other hand, does not absorb at that wavelength. It is therefore possible to monitor the formation of 4-nitrophenol at 25° C. by spectrophotometry with UV at 348 nm.

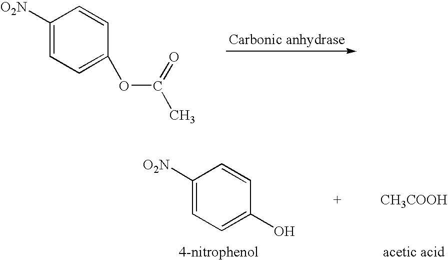

The hydrolysis reaction produced by carbonic anhydrase was therefore monitored at different concentrations by evolution of the absorbance or optical density (OD) of the reaction medium as a function of time at the wavelength specified above.

2. Preparation of the Reaction Medium

The enzyme used is bovine carbonic anhydrase (4.86 W.A.), extracted from erythrocytes, at a concentration of 0.0625 mg/ml (the unit W.A. is defined as follows: one W.A. causes the pH of the buffer solution Trizma to vary from 8.3 to 6.3 per minute at 0° C.).

The tertiary structures of the proteins (carbonic anhydrase) are subject to the influence of the pH. It is therefore evidently important to monitor the pH of the reaction medium during the hydrolysis kinetics.

The use of buffers such as Trizma/mercaptoethanol (di(tri(hydroxymethyl)aminomethane)sulfate) and sodium diethylmalonate helps to maintain the activity and stability of the enzyme.

The enzyme used (bovine carboanhydrase), the substrate (4-nitrophenyl acetate) and the other reagents (Trizma sulfate buffer, mercaptoethanol, sodium diethylmalonate) were supplied by Sigma Aldrich.

The study is based on the determination of the esterase activity of carbonic anhydrase.

The enzyme kinetics is effected by spectrophotometric monitoring (ultraviolet at 348 nm), allowing the evolution of the amounts of nitrophenol resulting from the hydrolysis of nitrophenyl acetate by carbonic anhydrase to be determined.

Preparation of the Enzyme

The enzyme is prepared in an aqueous solution composed of Trizma sulfate (0.05 M)/mercaptoethanol (1 mM) buffer.

The solution is adjusted to pH 8.7 by means of a pH meter using a 0.1N NaOH solution.

The concentration of enzyme used is 0.0625 mg of enzyme per ml of Trizma sulfate/mercaptoethanol buffer.

Preparation of the Substrate

The solution of substrate, 4-nitrophenyl acetate, was prepared daily, because that ester hydrolyzes weakly in light to give 4-para-nitrophenol and acetic acid. The solutions prepared are different according to the concentration of ester.

For weak concentrations (7.5×10⁻⁴ M to 1.875×10⁻⁴ M), the substrate was dissolved in a water/acetone mixture (v/v 98/2).

For strong concentrations (4.83×10⁻³ M; 3.28×10⁻³ M), the substrate was dissolved in a water/acetone/diethylmalonate buffer mixture (preparation below) in the relative proportions 13.7 ml/0.6 ml/5.7 ml, respectively.

Preparation of the Sodium Diethylmalonate Buffer

An aqueous solution of sodium diethylmalonate (0.12 M) adjusted to pH 7.2 is prepared.

Preparation of the Inhibitors

Different Types of Inhibitor were Tested.

The sodium salt of N-perfluorooctanesulfonyl: C₈F₁₇SO₂NH⁻⁺Na (M=521), dissolved in water, at concentrations ranging from 1.35×10⁻⁶ M to 1.35×10⁻⁸ M.

The other inhibitors used are not soluble in water without the addition of a co-solvent; DMSO at a concentration of 10% (V/V) was used.

The products below are dissolved in this aqueous DMSO solution.
acetazolamide (commercial inhibitor) at concentrations varying from 64×10⁻⁶ M to 1.35×10⁻⁸ M.
perfluorooctanesulfamide (C₈F₁₇SO₂NH₂) at concentrations ranging from 1.35×10⁻⁶ M to 1.35×10⁻⁸ M.
C₆F₁₃SO₂NH₂ at concentrations ranging from 1.35×10⁻⁶ M to 1.35×10⁻⁸ M.
NH₂SO₂C₄F₈SO₂NH₂ at concentrations ranging from 1.35×10⁻⁶ M to 1.35×10⁻⁸ M.
C₆H₁₃SO₂NH₂ at concentrations ranging from 1.35×10⁻⁴ M to 1.35×10⁻⁸ M.
C₈H₁₇SO₂NH₂ at concentrations ranging from 1×10⁻⁴ M to 1.35×10⁻⁸ M.

3) Study of the Hydrolysis of Para-Nitrophenyl Acetate in the Absence of Inhibitor

Procedure of the Enzyme Kinetics

Hydrolysis Kinetics of the Substrate Without Inhibitor

The following were introduced into the spectrophotometer chamber:
0.2 ml of sodium diethylmalonate buffer
0.2 ml of 4-nitrophenyl acetate
0.3 ml of milliQ water
0.1 ml of enzyme prepared in the buffer as described hereinbefore: the enzyme addition corresponds to time zero of the kinetics.

Hydrolysis Kinetics of the Substrate with Inhibitor

The following are introduced into the spectrophotometer chamber:
0.2 ml of sodium diethylmalonate buffer
0.3 ml of inhibitors
0.1 ml of enzyme.

The whole is incubated for 5 minutes, and then the hydrolysis kinetics is initiated by addition of 0.2 ml of 4-nitrophenyl acetate.

First of all, in order to validate the study protocol of carbonic anhydrase, the hydrolysis of NPA in the absence of inhibitor for an enzyme concentration of 2.1×10⁻⁶ M was studied. An excess of substrate relative to the concentration of enzyme was used: this is a required Michaelian condition.

The substrate concentrations studied are 4.83×10⁻³ M; 7.5×10⁻³ M; 4.5×10⁻⁴ M; 3.75×10⁻⁴ M; 2.625×10⁻⁴ M; 1.875×10⁻⁴ M.

a) Determination of the Initial Rates of the Hydrolysis Reaction of the Substrate by the Enzyme

For each substrate concentration, the graphs showing the OD as a function of time allowed the initial reaction rate to be calculated.

b) Results

After the initial rates (Vo) had been obtained for each substrate concentration, two secondary graph types were used to extract the kinetic constants Km (Michaelis constant) and Vm (maximum reaction rate).

Lineweaver-Burk Graph

The first representation is that of Lineweaver-Burk, based on the equation:

$$\frac{1}{v} = \frac{K m + [ S ]}{V m [ S ]} = \frac{K m}{V m} \times \frac{1}{[ S ]} + \frac{1}{V m}$$

The intersection of the straight line with the X-axis allowed −1/Km to be determined, from which Km=8.10⁻³ mol.l⁻¹.

The intersection of the straight line with the Y-axis allowed 1/Vm to be determined, from which Vm=2.3 mol.l⁻¹.min⁻¹.

These results are confirmed by analysis of the Eadie-Hoffstee graph.

The results obtained for Km and Vm from those two graphs are of the same order of magnitude.

Those results made it possible to conclude that the enzyme has high affinity for its substrate (NPA).

4. Study of the Inhibiting Effect of Fluorinated Sulfamides

a) Inhibiting Effect of Acetazolamide

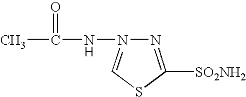

In order to calibrate the inhibiting effect of the products, acetazolamide was chosen as reference (active ingredient of Diamox). Acetazolamide is a non-competitive carbonic anhydrase inhibitor.

In order to assess the inhibiting role of acetazolamide, solutions having different concentrations ranging from 64 μmol/l to 0.0135 μmol/l were prepared, the term μmol/l referring, within the context of this description, to micromoles/litre, which is equivalent to 10⁻⁶ mol/l.

TABLE 1
Inhibition of the hydrolysis of p-nitrophenyl acetate, at a concentration
of 7.5 × 10⁻⁴ M, by carbonic anhydrase at a
concentration of 0.0625 mg/ml in the presence of
increasing concentrations of acetazolamide

Acetazolamide
concentrations in μM
(μmol/l)

| | 64 | 16 | 3.2 | 1.6 | 1.3 | 0.0135 |
|---|---|---|---|---|---|---|
| Total inhibition | yes | yes | yes | yes | yes | no |

It was therefore verified that at a concentration of 1.35×10⁻⁶ M, the molecules also exhibit an inhibiting effect.

In order to facilitate comparison between the various molecules, the concentration of 1.35×10⁻⁶ moles was therefore chosen as the test concentration of the sulfamides which were synthesized (C₆F₁₃SO₂NH₂, C₈F₁₇SO₂NH₂, C₈F₁₇SO₂NH⁻Na⁺, (C₂F₄SO₂(NH₂))₂, C₆H₁₃SO₂NH₂, C₈H₁₇SO₂NH₂).

b) Inhibiting Effect of Fluorinated Sulfamides

The inhibition of carbonic anhydrase by the compounds of formula (I) was tested under the same conditions at a concentration of 1.35×10⁻⁶ M and compared with acetazolamide and with the hydrogenated sulfamides.

TABLE 2
% inhibition of the hydrolysis of nitrophenyl acetate (NPA)
catalyzed by carbonic anhydrase at 25° C.

| | | Concentration | | | |
|---|---|---|---|---|---|
| | | mol/l | | | |
| | | 1.35 | 1.35 | 1.35 | 1.35 |
| | Inhibitor | 10⁻⁴ | 10⁻⁶ | 10⁻⁷ | 10⁻⁸ |
| Perfluorinated | C₈F₁₇SO₂NH⁻⁺Na | | 94 | 40 | 17 |
| sulfamides | C₈F₁₇SO₂NH⁻⁺Li | | 85 | 3 | |
| | C₈F₁₇SO₂NH₂ | | 88 | 7 | 5 |
| | C₇F₁₅SO₂NH₂ | | 80 | 0 | |
| | C₆F₁₃SO₂NH₂ | | 80 | 2 | |
| Fluorohydro- | C₈F₁₇(CH₂)₂SO₂NH₂ | | 36 | | |
| genated | C₆F₁₃(CH₂)₂SO₂NH₂ | | 66 | | 1 |
| sulfamides | | | | | |
| Double | (C₂F₄SO₂NH₂)₂ | | 78 | 6 | 1 |
| sulfamide | | | | | |
| Hydrogenated | C₈H₁₇SO₂NH₂ | 86 | 5 | | |
| sulfamides | C₆H₁₃SO₂NH₂ | 73 | 0 | | |
| Commercial | Acetazolamide | | 85 | 52 | 6 |
| product | | | | | |
| Sulfinimides | (C₆F₁₃SO₂)₂NH | 85 | 0 | | |
| | (C₈F₁₇SO₂)₂NH | 89 | 0 | | |
| | (C₄F₉SO₂)₂NH | 65 | 0 | | |
| | (C₈F₁₇SO₂)NH(C₆F₁₃SO₂) | 87 | 0 | | |
| | (C₆F₁₃SO₂)NH(C₄F₉SO₂) | 87 | 0 | | |
| | (C₄F₉SO₂)NH(C₈F₁₇SO₂) | 97 | 0 | | |

The results show that the compounds of formula (I) have an inhibiting effect with respect to carbonic anhydrase. It will be noted in particular that the four fluorinated products (C₆F₁₃SO₂NH₂, C₈F₁₇SO₂NH₂, C₈F₁₇SO₂NH⁻Na⁺(C₂F₄SO₂(NH₂)₂) exhibit very considerable inhibition with respect to carboanhydrase, comparable with or even superior to that of acetazolamide. Interestingly, it should be noted that this inhibiting activity is all the greater as the number of fluorine atoms increases. Thus, the products having a C₈ fluorinated chain (C₈F₁₇SO₂NH₂ and C₈F₁₇SO₂NH⁻Na⁺) are more active than the compounds having a C₆ chain (C₆F₁₃SO₂NH₂), which are themselves more active than the compound having a C₄ chain (C₂F₄SO₂(NH₂)₂).

These results show that fluorine plays a significant part in inhibiting carbonic anhydrase.

In fact, a study of the hydrogenated homologues (C₆H₁₃SO₂NH₂, C₈H₁₇SO₂NH₂) under the same conditions clearly shows, in a series of molecules, that replacing the hydrogen atoms by fluorine atoms permits a substantial increase in the inhibitory nature.

1-24. (canceled)

25. Pharmaceutical composition comprising compounds of formula (I):
NZ₁Z₂Z₃ (I)
in which:
Z₁, Z₂, Z₃ each independently of the others represents:
a hydrogen atom;
a C₁-C₆-alkyl group;
a group —SO₂R₃ wherein R₃ represents a linear or branched C₁-C₁₂-alkyl, -alkenyl or -alkynyl group, a C₃-C₁₀-cycloalkyl group or a C₆-C₁₀-aryl group, a (C₁-C₆)-alkyl-(C₆-C₁₄)-aryl group, or a C₅-C₁₀-heteroaryl group;
it being understood that at least one of the groups Z₁, Z₂, Z₃ represents a group of formula (II)
X—R_{F}—(CH₂)ₙ—SO₂— (II)
in which
X represents a hydrogen atom; a fluorine atom; a group —SO₂NR₁R₂ wherein R₁, R₂ may be identical or different and each independently of the other represents a hydrogen atom, a C₁-C₆-alkyl group, a pharmaceutically acceptable cation selected from the alkali metal or alkaline earth metal cations, ammonium or protonated or quaternized amines; or a group —SO₂—R₃;
R_{F} represents a linear or branched, poly- or per-fluorinated C₁-C₁₂-alkylene group;
n represents an integer from 0 to 6, it being understood that when n=0, one of the groups Z₁, Z₂, Z₃ may further represent a pharmaceutically acceptable cation selected from the alkali metal or alkaline earth metal cations, ammonium or protonated or quaternized amines;
and the pharmaceutically acceptable salts of those compounds;
with the exception of
compounds of formula (I) in which n=0 and at least one of the groups Z₁, Z₂, Z₃ represents CF₃SO₂; and
the compound (C₈F₁₇)SO₂NH(C₂H₅).

26. Pharmaceutical composition according to claim 25, in which Z₁, Z₂, Z₃ each independently of the others represents
a hydrogen atom;
a group —SO₂R₃;
it being understood that at least one of the groups Z₁, Z₂, Z₃ represents a group of the formula X—R_{F}—(CH₂)ₙ—SO₂—,
R₃, X, R_{F}, n being as defined in claim 1.

27. Pharmaceutical composition according to claim 25, in which Z₂ represents a hydrogen atom.

28. Pharmaceutical composition according to claim 25, in which Z₃ represents a hydrogen atom.

29. Composition according to claim 25, in which X represents a fluorine atom.

30. Composition according to claim 25, in which R_{F} represents a linear poly- or per-fluorinated alkylene group.

31. Composition according to claim 30, in which R_{F} represents a perfluorinated alkylene group.

32. Composition according to claim 25, in which R_{F} represents a C₆-C₁₂-alkylene group, preferably a C₆-C₈-alkylene group.

33. Composition according to claim 25, in which n=2.

34. Composition according to claim 25, in which X represents a group —SO₂NR₁R₂.

35. Composition according to claim 25, in which R₁ and R₂ represent a hydrogen atom.

36. Composition according to claim 34, in which R_{F} represents a perfluorinated C₂- to C₆-alkylene radical.

37. Pharmaceutical composition according to claim 25, in which n=0.

38. Pharmaceutical composition according to claim 37, in which Z₃ represents a pharmaceutically acceptable cation selected from the alkali metal or alkaline earth metal cations, ammonium or protonated or quaternized amine.

39. Pharmaceutical composition according to claim 38, in which the alkali metal or alkaline earth metal cation is selected from the sodium, potassium, magnesium and lithium ions.

40. Pharmaceutical composition according to claim 25, in which Z₁ and Z₂, which may be identical or different, each represents a group of formula (II).

41. Composition according to claim 40, in which Z₁ and Z₂ are identical.

42. Composition according to claim 25, in which Z₂ represents a group —SO₂R₃.

43. Composition according to claim 25, in which the compound of formula (I) contains at least 10 fluorine atoms.

44. Composition according to claim 25, in which the compounds of formula (I) are:
C₈F₁₇SO₂NH⁻⁺Na
C₈F₁₇SO₂NH⁻⁺Li
C₈F₁₇SO₂NH₂
C₇F₁₅SO₂NH₂
C₆F₁₃SO₂NH₂
C₈F₁₇(CH₂)₂SO₂NH₂
C₆F₁₃(CH₂)₂SO₂NH₂
(C₂F₄SO₂NH₂)₂
(C₆F₁₃SO₂)₂NH
(C₈F₁₇SO₂)₂NH
(C₄F₉SO₂)₂NH
(C₈F₁₇SO₂)NH(C₆F₁₃SO₂)
(C₆F₁₃SO₂)NH(C₄F₉SO₂)
(C₄F₉SO₂)NH(C₈F₁₇SO₂)

45. A method for the treatment of pathologies in which metallo-enzyme activity is involved, comprising administering an effective amount of a composition according to claim 25 to a subject in need thereof.

46. The method according to claim 45, in which the metallo-enzymes are selected from carbonic anhydrase, butolic toxin, anthrax toxin, tetanic toxin, bacterial elastase, integrase, and angiotensin converting enzyme, and the lethal factor of carbon.

47. The method according to claim 45, in which the pathologies are selected from glaucoma, tetanus, botulism, anthrax, mucoviscidosis, superinfections, AIDS, cardiovascular diseases.

48. The method according to claim 47 wherein the subject suffers from glaucoma.
